(19) United States
(12) Patent Application Publication
(10) Pub. No.: US 2006/0135630 A1
(43) Pub. Date: Jun. 22, 2006

(54) Producing longer-chain hydrocarbons from natural gas

(76) Inventor: Michael Joseph Bowe, OXFORDSHIRE (GB)

Correspondence Address: LAW OFFICES OF WILLIAM H. HOLT, 12311 HARBOR DRIVE, WOODBRIDGE, VA, 22192, US

(21) Appl. No.: 10/543,410

(22) PCT Filed: Feb. 25, 2004
(86) PCT No.: PCT/GB04/00738

§ 371 Date: Jul. 26, 2005

(51) Int. Cl. C07C 27/06

(52) U.S. Cl. 518/702; 518/703

(57) ABSTRACT

Natural gas is reacted with steam, to generate carbon monoxide and hydrogen in a first catalytic reactor (16); the resulting gas mixture is used to perform Fischer-Tropsch synthesis in a second catalytic reactor (26). After cooling the resulting gas stream in a heat exchanger (29), the remaining gas stream (30) is cooled a second time by decreasing its pressure (32), and the resulting condensed liquid is separated (33) from the remaining gas phase. This increases the production of condensate. The second cooling step may utilise a turbine (32), or a vortex tube (40), or a throttle valve.

This invention relates to a chemical process, and to a plant including catalytic reactors suitable for use in performing the process, for producing longer-chain hydrocarbons from natural gas.

A process is described in WO 01/51194 (Accentus plc) in which methane is reacted with steam, to generate carbon monoxide and hydrogen in a first catalytic reactor; the resulting gas mixture is then used to perform Fischer-Tropsch synthesis in a second catalytic reactor. The overall result is to convert methane to hydrocarbons of higher molecular weight, which are usually liquid under ambient conditions. The two stages of the process, steam/methane reforming and Fisher-Tropsch synthesis, require different catalysts, and catalytic reactors are described for each stage. The catalytic reactors enable heat to be transferred to or from the reacting gases, respectively, as the reactions are respectively endothermic and exothermic; the heat required for steam/methane reforming may be provided by combustion. However the proportions of carbon monoxide and hydrogen produced by the steam/methane reforming are not ideal for the Fischer-Tropsch synthesis. An improved way of performing this overall process has now been found.

According to the present invention there is provided a process for processing natural gas to generate longer-chain hydrocarbons, the process comprising subjecting the gas to steam reforming to generate a mixture of carbon monoxide and hydrogen, then subjecting this mixture to Fischer-Tropsch synthesis at an elevated pressure, and subjecting the gas stream resulting from the Fischer-Tropsch synthesis to a first cooling process by heat exchange to condense at least some of the products of the Fischer-Tropsch synthesis, wherein the remaining gas stream is then subjected to a second cooling process to cool it by decreasing its pressure, and the resulting condensed liquid is separated from the remaining gas phase.

The second cooling process will cause condensation of more of the products, and the resulting liquid may be combined with the condensed products from the first cooling process. The remaining gases are preferably supplied to a combustion channel to provide heat for the endothermic steam-reforming reaction.

The present invention also provides a plant for processing natural gas to generate longer-chain hydrocarbons, the plant comprising a reforming reactor for subjecting the gas to steam reforming to generate a mixture of carbon monoxide and hydrogen, a reactor for subjecting this mixture to Fischer-Tropsch synthesis at elevated pressure, and a heat exchanger to cool the gas stream resulting from the Fischer-Tropsch synthesis, the plant also comprising a second cooling means wherein the remaining gas stream is cooled by decreasing its pressure, and means to separate resulting condensed liquid from the remaining gas phase after the second cooling means.

Each reactor is preferably a compact catalytic reactor, containing a catalyst appropriate to the reaction (i.e. reforming, and Fischer-Tropsch synthesis respectively). Each reactor preferably comprises a plurality of flat metal sheets in a stack, with grooves which define first and second gas flow channels, the channels being arranged alternately to ensure good thermal contact between the gases in them. Appropriate catalysts are provided in those channels in which a reaction is to occur, depending on the required reaction. To ensure the required good thermal contact, in the reforming reactor both the first and the second gas flow channels are preferably less than 5 mm in the direction normal to the sheets, more preferably less than 3 mm deep, while in the Fischer-Tropsch reactor the reaction channels are preferably less than 10 mm deep. Corrugated or dimpled metallic foils, metal meshes, or corrugated or pleated metal felt sheets may be used as the substrate of a catalyst structure within the flow channels to enhance heat transfer and catalyst surface area. These catalyst structures are preferably removable from the grooves in the stack, so they can be replaced if the catalyst becomes spent.

The heat exchanger preferably causes the gas stream to exchange heat with a fluid at ambient temperature, so that active refrigeration is not required. For example, if the plant is on a floating platform at sea, then the heat exchanger would preferably entail heat exchange with seawater. This typically would be of temperature in the range 5° C. up to 25° C., depending on geographical location and time of year.

To maximise the yield of desired products, which are typically C5 and above, further cooling is beneficial. Since the Fischer-Tropsch synthesis typically occurs at a pressure of about 2 MPa, advantage can be taken of this. The secondary cooling may be achieved by using a Joule Thomson valve in which the gases expand without any transfer of heat from the surroundings into a lower pressure state; such a valve may be referred to as a throttling valve. Alternatively the secondary cooling may utilise a vortex tube separator; this incorporates Joule-Thomson cooling at the inlet nozzles, with further cooling in the vortex section. Both the Joule Thomson valve and the vortex tube separator have the benefit of no moving parts, and the vortex tube separator can produce a greater cooling effect. A further possibility is to use a turbine, preferably a two-phase turbine, so that the gases not only expand but also do work, and consequently are cooled even more.

The invention will now be further and more particularly described, by way of example only, and with reference to the accompanying drawings, in which:

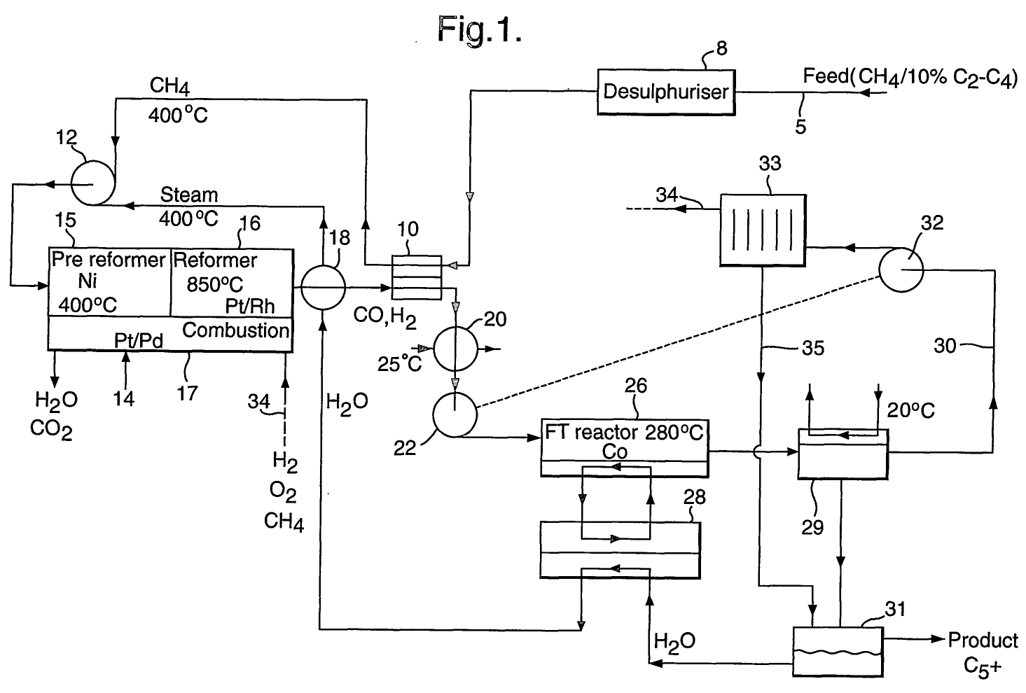

FIG. 1 shows a flow diagram of a chemical process of the invention; and

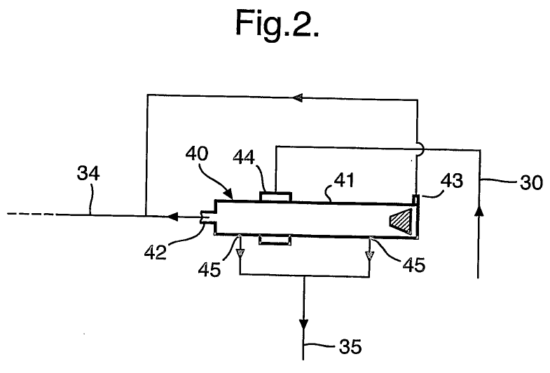

FIG. 2 shows a modification of part of the flow diagram of FIG. 1.

The invention relates to a chemical process for converting natural gas (primarily methane) to longer chain hydrocarbons. The first stage involves steam reforming, that is to say the reaction of the type:
H₂O+CH₄→CO+3H₂
This reaction is endothermic, and may be catalysed by a rhodium or platinum/rhodium catalyst in a first gas flow channel in a catalytic reactor. The heat required to cause this reaction may be provided by combustion of an inflammable gas such as methane or hydrogen, which is exothermic and may be catalysed by a palladium catalyst in an adjacent second gas flow channel. Such flow channels may be defined in successive metal sheets in a stack. In both cases the catalyst is preferably on a stabilised-alumina support which forms a coating typically less than 100 μm thick on a metallic substrate.

Both these reactions may take place at atmospheric pressure, although alternatively the reforming reaction might take place at an elevated pressure. The heat generated by the combustion would be conducted through the metal sheet separating the adjacent channels.

The gas mixture produced by the steam/methane reforming can then be used to perform a Fischer-Tropsch synthesis to generate a longer chain hydrocarbon, that is to say:
nCO+2nH₂→(CH₂)ₙ+nH₂O
which is an exothermic reaction, occurring at an elevated temperature, typically between 200 and 300° C., for example 220° C., and an elevated pressure typically between 2 MPa and 4 MPa, for example 2.5 MPa, in the presence of a catalyst. The exact nature of the organic compounds formed by the reaction depends on the temperature, the pressure, and the catalyst, as well as the ratio of carbon monoxide to hydrogen. The heat given out by this synthesis reaction may be used to provide at least part of the heat required by the steam/methane reforming reaction, for example a heat transfer fluid may be used to transfer the heat from a reactor in which the Fischer-Tropsch synthesis is occurring, the heat being used to preheat at least one of the gas streams supplied to the reforming reactor. The preferred catalyst for the Fischer-Tropsch synthesis comprises a coating of gamma-alumina of specific surface area 140-230 m²/g with about 10-40% cobalt (by weight compared to the alumina), and with a ruthenium, platinum or gadolinium promoter which is less than 10% the weight of the cobalt.

Referring now to the drawing, the overall chemical process is shown as a flow diagram. The feed gas 5 is natural gas consisting primarily of methane, with a small percentage (say 10%) of ethane and propane. It is passed through a heat exchanger 10 so it is at about 400° C. and is then supplied via a fluidic vortex mixer 12 to a first catalytic reactor 14; in the mixer 12 the feed gas is mixed with a stream of steam that is also at about 400° C., the streams entering the mixer 12 through tangential inlets and following a spiral path to an axial outlet so they become thoroughly mixed. Both streams may be at atmospheric pressure, or for example at a pressure of say 100 kPa above atmospheric. The flows are preferably such that the steam: methane molar ratio (at the steam/methane reforming stage) is between 1.3 and 1.6, preferably 1.4 to 1.5. The first part of the reactor 14 is a pre-reformer 15 with a nickel or platinum/rhodium methanation catalyst, in which the higher alkanes react with the steam to form methane (and carbon monoxide); extra steam is evidently required to ensure the specified steam/methane ratio is achieved. This pre-reformer 15, which may be a separate reactor, would not be required if the feed gas 5 contained substantially no higher alkanes. The second part of the reactor 14 is a reformer 16 with a platinum/rhodium catalyst, in which the methane and steam react to form carbon monoxide and hydrogen. The temperature increases gradually from about 350° C. at the start of the pre-reformer 15 to about 850° C. in the reformer 16.

If the feed gas 5 contains any sulphur-containing compounds it is passed through a desulphurisation unit 8 before reaching the first catalytic reactor 14. As shown in the flow diagram this unit 8 may treat the gas at its initial temperature, for example using liquid scrubbing absorption. Alternatively, it may use a desulphurisation technique that requires elevated temperatures, for example a solid state absorption process, and instead be arranged to treat the gas 5 after it has passed through the heat exchanger 10.

The heat for the endothermic reactions in the reactor 14 may be provided by combustion (e.g. of methane or hydrogen) over a palladium or platinum catalyst within adjacent gas flow channels 17. The catalyst may include gamma-alumina as a support, coated with a palladium/platinum 3:1 mixture, which is an effective catalyst over a wide temperature range. The combustible gas mixture preferably flows in counter-current to the gases in the reformer 16. The combustible gas mixture may be supplied in stages along the reactor 14, to ensure combustion occurs throughout its length.

The hot mixture of carbon monoxide and hydrogen emerging from the reformer 16 is then quenched by passing through a heat exchanger 18 to provide the hot steam supplied to the vortex mixer 12, and then through the heat exchanger 10 in which it loses heat to the feed gas 5. The mixture is then further cooled to about 100° C. by passing through a heat exchanger 20 cooled by water. The gases are then compressed through a compressor 22 to a pressure of 2.5 MPa (25 atm.).

The stream of high pressure carbon monoxide and hydrogen is then supplied to a catalytic reactor 26 in which the gases react, undergoing Fischer-Tropsch synthesis to form a paraffin or similar compound. This reaction is exothermic, preferably taking place at about 220° C., and the heat generated may be used to preheat the steam supplied to the heat exchanger 18, using a heat exchange fluid circulated between heat exchange channels in the reactor 26 and a steam generator 28. During this synthesis the volume of the gases decreases. The resulting gases are then passed into a condenser 29 in which they exchange heat with water initially at ambient temperature of say about 20° C. The higher alkanes (say C5 and above) condense as a liquid, as does the water, this mixture of liquids being passed to a gravity separator 31; the separated higher alkanes can then be removed as the desired product, while the water is returned via the heat exchangers 28 and 18 to the mixer 12.

It will be appreciated, from the equations discussed above, that the steam reforming stage forms more hydrogen than is required for the Fischer-Tropsch synthesis. Consequently the gas phase emerging from the condenser 29 through the outlet pipe 30 contains a significant quantity of hydrogen, as well as lower alkanes (say C1 to C5), and a proportion of the higher alkanes which have not condensed.

This gas mixture is passed through a pressure-reducing two-phase turbine 32 (for example a two-phase impulse turbine) and then fed to a de-entraining separator 33 in which condensed liquids are separated from the remaining gas stream. The separator 33 contains an array of vertical impactor plates. As the gas mixture passes through the turbine 32 it loses pressure and at the same time does work, so its temperature drops. The gas stream is fed through the pipeline 34 to the catalytic combustion channels 17 of the reformer 14, being combined with air to provide the necessary oxygen. The condensed liquids are returned through a pipe 35 to the gravity separator 31, so increasing the amount of higher molecular weight products.

As indicated by the chain-dotted line, electricity generated by the turbine 32 may be used to help drive the compressor 22.

Feeding the hydrogen-rich gas stream into the combustion channel 17 of the reactor 14 has been found to give a more uniform temperature distribution, and also enables the combustion reaction to be initiated more readily when the reactor is cold (as catalytic combustion can then occur at a temperature as low as 15 or 20° C.). The overall thermal efficiency of the process is improved, the amount of methane from the feed gas 5 fed directly to the combustion channels is decreased or possibly reduced to zero, and the emission of carbon dioxide to the environment is reduced.

Although the two-phase turbine 32 is effective at bringing about additional cooling, it has the complexity of moving parts, and so the need of maintenance. In an alternative the turbine 32 may be replaced by a vortex tube, as shown in FIG. 2, which shows a modification to a part of the flow diagram of FIG. 1. As shown in FIG. 2, a vortex tube 40 comprises a tubular duct 41 with an axial outlet 42 at one end and a peripheral gas outlet 43 at the other end, the high pressure gas flow from the pipe 30 being supplied through tangential nozzles 44 into the duct 41. The gas cools as it passes through the inlet nozzles 44, and then as a result of the forced vortex within the tubular duct is separated into warm and cold gas fractions which emerge through opposite ends of the duct 41. The overall process is effectively isentropic. The vortex flow within the tubular duct 41 causes liquid droplets to impact with the wall, so the liquid phase is separated from the gas phase by the vortex tube itself, which has separate outlets 45 for condensed liquid. The condensed liquid emerging from the outlets 45 is supplied to the pipe 35. The resulting gas streams are recombined before being supplied through the pipeline 34 to the combustion channel 17.

As an even greater simplification, the turbine 32 in FIG. 1 might be replaced by a throttle valve, so that the gases are cooled by the Joule Thomson effect. This is a considerably simpler device even compared to the vortex tube, but is the least effective at cooling the gases.

In a further modification, the de-entraining separator 33 (whether used in conjunction with a turbine 32 or a throttle valve) may be replaced by a different device to separate liquid droplets from the gas stream, for example swirl tubes, a demister pad, or a stack of corrugated plates.

1. A process for processing natural gas (5) to generate longer-chain hydrocarbons, the process comprising subjecting the gas to steam reforming (16) to generate a mixture of carbon monoxide and hydrogen, then subjecting this mixture to Fischer-Tropsch synthesis (26) at an elevated pressure, and subjecting the gas stream resulting from the Fischer-Tropsch synthesis to a first cooling process (29) by heat exchange to condense at least some of the products of the Fischer-Tropsch synthesis, characterized in that the remaining gas stream is then subjected to a second cooling process (32; 40) to cool it by decreasing its pressure, and the resulting condensed liquid is separated (33, 35) from the remaining gas phase (34).

2. A process as claimed in claim 1 wherein the remaining gases are supplied to a combustion channel (17) to provide heat for the endothermic steam-reforming reaction (16).

3. A plant for processing natural gas (5) to generate longer-chain hydrocarbons, the plant comprising a reforming reactor (16) for subjecting the gas to steam reforming to generate a mixture of carbon monoxide and hydrogen, a reactor (26) for subjecting this mixture to Fischer-Tropsch synthesis at elevated pressure, and a heat exchanger (29) to cool the gas stream resulting from the Fischer-Tropsch synthesis, characterized by the plant also comprising a second cooling means (32; 40) wherein the remaining gas stream is cooled by decreasing its pressure, and means (33) to separate resulting condensed liquid from the remaining gas phase (34) after the second cooling means.

4. A plant as claimed in claim 3 wherein each reactor is a compact catalytic reactor, containing a catalyst appropriate to the reaction.

5. A plant as claimed in claim 3 wherein the second cooling means comprises a turbine (32).

6. A plant as claimed in claim 3 wherein the second cooling means comprises a vortex tube (40).

7. A plant as claimed in claim 6 wherein the vortex tube (40) incorporates a separation means (45) for the condensed liquid.

8. A plant as claimed in claim 3 wherein the second cooling means comprises a throttle valve.
